(19) United States
(12) Patent Application Publication
(10) Pub. No.: US 2022/0380489 A1
(43) Pub. Date: Dec. 1, 2022

(54) METHOD FOR PRODUCING HEPARIN-LIKE SUBSTANCE, RECOMBINANT CELL, AND METHOD FOR PRODUCING THE SAME

(71) Applicants: KYUSHU UNIVERSITY, NATIONAL UNIVERSITY CORPORATION, Fukuoka-shi, Fukuoka (JP); NATIONAL UNIVERSITY CORPORATION CHIBA UNIVERSITY, Chiba-shi, Chiba (JP); KYOWA HAKKO BIO CO., LTD., Tokyo (JP)

(72) Inventors: Masamichi KAMIHIRA, Fukuoka (JP); Yoshinori KAWABE, Fukuoka (JP); Kosuke SAGAWA, Fukuoka (JP); Toshihiko TOIDA, Chiba (JP); Satoshi KOIZUMI, Tokyo (JP)

(73) Assignees: KYUSHU UNIVERSITY, NATIONAL UNIVERSITY CORPORATION, Fukuoka-shi, Fukuoka (JP); NATIONAL UNIVERSITY CORPORATION CHIBA UNIVERSITY, Chiba-shi, Chiba (JP); KYOWA HAKKO BIO CO., LTD., Tokyo (JP)

(21) Appl. No.: 17/765,507

(22) PCT Filed: Oct. 2, 2020
(86) PCT No.: PCT/JP2020/037626

§ 371 Date: Mar. 31, 2022

(30) Foreign Application Priority Data
Oct. 2, 2019 (JP) 2019-182467

(51) Int. Cl. C08B 37/00; C12N 15/63; C12N 5/071; C12N 5/10

(52) U.S. Cl. CPC C08B 37/0075; C12N 15/63; C12N 5/0682; C12N 5/10

(57) ABSTRACT

An object of the present invention is to provide a method for efficiently producing a heparin-like substance without using an animal-derived tissue. The present invention relates to a method for producing a heparin-like substance and the like, the method comprising: (1) preparing a mammalian cell that produces a heparin-like substance, (2) preparing a recombinant cell in which a gene that encodes an extracellular domain of syndecan is introduced into the mammalian cell that produces a heparin-like substance and is prepared in step (1), and (3) culturing the recombinant cell prepared in step (2) in a medium and collecting the heparin-like substance from the resulting culture supernatant.

### TECHNICAL FIELD

The present invention relates to a method for producing a heparin-like substance, a recombinant cell, and a method for producing the same.

### BACKGROUND ART

Heparin is an essential drug that is used in medical practice such as artificial dialysis and extracorporeal circulation as an anticoagulant. Heparin is used in an injectable form, for example, as a prophylactic or therapeutic agent for thromboembolism. Heparin is also used to form an anticoagulant surface on various experimental and medical devices such as a kidney dialysis machine.

Heparin is a polysaccharide that is one of four major subfamilies of glycosaminoglycans (hereinafter also abbreviated as GAG). GAG is a long polysaccharide composed of a repetitive disaccharide unit. GAG is mainly classified into four subfamilies: (1) heparan sulfate (hereinafter also abbreviated as HS)/heparin, (2) keratan sulfate (KS), (3) chondroitin/dermatan sulfate (CS/DS), and (4) hyaluronic acid or hyaluronan (HA).

The subfamilies of GAG differ from each other in a monosaccharide component, a glycosidic linkage, and a sugar position and degree. Except for HA, GAG is covalently bound to a core protein, and GAG covalently bound to the core protein is known as proteoglycan.

Heparin and HS are heterogeneous hybrids of acidic mucopolysaccharides, and heparin is a type of HS. Heparin/HS is a linear polysaccharide comprising a disaccharide of uronic acid (β-D-glucuronic acid and α-L-iduronic acid) and glucosamine (DN-acetylglucosamine and DN-glucosamine sulfate) as one unit, those disaccharides being linked by tens to hundreds of α or β-1,4 linkages. A molecular weight varies from 3 kDa to 30 kDa depending on a difference in a chain length, with an average of 12 kDa to 15 kDa. Examples of modification of heparin/HS include O-sulfation of uronic acid at 2-position, O-sulfation of glucosamine at 3-position and 6-position, and N-sulfation of an amino group at 2-position.

Almost all mammalian cells produce GAGs, which are incorporated into proteoglycans in cell-associated sugar chains present in an extracellular matrix to define tissue morphology and function. HS regulates growth and generation of cells by regulating a growth factor such as a fibroblast growth factor (FGF) family, a platelet-derived growth factor (PDGF), and a vascular endothelial cell growth factor (VEGF). On the other hand, heparin is mainly produced in mast cells.

Heparin used in pharmaceutical products and the like is mainly extracted and purified from a small intestine of a pig or a lung of a bovine. However, heparin obtained from these supply sources has been associated with several adverse events due to contamination with persulfated chondroitin sulphate (Non-Patent Document 1). In addition, the isolation of heparin from animal tissues has a risk of propagation of pathogens such as viruses, bacteria, and transmissible spongiform encephalopathy from animals to humans Therefore, development of a new technology for producing heparin without using an animal-derived tissue is desired. Although heparin can be synthesized by a chemical or chemoenzymatic method, it is difficult to produce a heterogeneous structural population present in animal-derived heparin.

On the other hand, syndecan is a family of four proteoglycans on a cell surface with a retained plasma membrane domain and a cytoplasmic domain. Covalently bound glycosaminoglycan chains are present in an extracellular domain. The covalently bound glycosaminoglycan chains are mainly heparan sulfate (HS), and also include chondroitin sulfate (CS) (Non-Patent Document 2).

### RELATED ART

### Patent Document

### Non-Patent Document

[Non-Patent Document 1] Guerrini et al, Nat. Biotechnol., 2008, 26, pp. 669-675

[Non-Patent Document 2] Trends in Glycoscience and Glycotechnology, 1993, Vol. 5, No. 22, pp. 107-120

### DISCLOSURE OF INVENTION

### Technical Problem

### Problems to Be Solved by the Invention

As described above, development of a new technology for producing heparin without using an animal-derived tissue is desired. The present inventors have attempted to produce a heparin-like substance using a mammalian cell that produces a heparin-like substance, and found that proteoglycan containing a heparin-like substance produced from the mammalian cell adheres to a cell membrane and is less likely to be secreted into a culture supernatant. Therefore, a complicated purification process is required, which is a major problem in industrial production of a heparin-like substance. Accordingly, an object of the present invention is to provide a production method for efficiently producing a heparin-like substance without using an animal-derived tissue.

### Means for Solving the Problems

The present inventors have found that the above problems can be solved by introducing a gene that encodes an extracellular domain of syndecan into a mammalian cell that produces a heparin-like substance, and have completed the present invention.

The present invention is as follows.

1. A method for producing a heparin-like substance, the method comprising the following steps (1) to (3):

(1) preparing a mammalian cell that produces a heparin-like substance;

(2) preparing a recombinant cell in which a gene that encodes an extracellular domain of syndecan (hereinafter abbreviated as SDC) is introduced into the mammalian cell that produces a heparin-like substance and is prepared in step (1); and

(3) culturing the recombinant cell prepared in step (2) in a medium, and collecting a heparin-like substance from a resulting culture supernatant.

2. The production method according to 1, in which the mammalian cell that produces a heparin-like substance is a cell into which at least one of a gene that encodes bifunctional heparan sulfate ester N-deacetylase/N-sulfate transferase (hereinafter abbreviated as NDST2) and a gene that encodes heparan sulfate ester glucosamine 3-sulfate transferase 1 (hereinafter abbreviated as Hs3st1) is introduced.
3. The production method according to 1 or 2, in which the mammalian cell is a Chinese hamster-derived ovary (hereinafter abbreviated as CHO) cell.
4. The production method according to 2 or 3, in which NDST2 is a protein comprising any one of the following amino acid sequences (1a) to (1c) and having a function of NDST2:

(1a) the amino acid sequence of SEQ ID NO: 2,

(1b) an amino acid sequence in which one to several amino acids are deleted, substituted or added in the amino acid sequence of SEQ ID NO: 2, and

(1c) an amino acid sequence having 80% or more homology with the amino acid sequence of SEQ ID NO: 2.

5. The production method according to any one of 2 to 4, in which Hs3st1 is a protein comprising any one of the following amino acid sequences (2a) to (2c) and having a function of Hs3st1:

(2a) the amino acid sequence of SEQ ID NO: 3,

(2b) an amino acid sequence in which one to several amino acids are deleted, substituted or added in the amino acid sequence of SEQ ID NO: 3, and

(2c) an amino acid sequence having 80% or more homology with the amino acid sequence of SEQ ID NO: 3.

6. The production method according to any one of 1 to 5, in which the extracellular domain of SDC is a protein comprising any one of the following amino acid sequences (3a) to (3c) and having a function of the extracellular domain of SDC:

(3a) the amino acid sequence of SEQ ID NO: 1,

(3b) an amino acid sequence in which one to several amino acids are deleted, substituted or added in the amino acid sequence of SEQ ID NO: 1, and

(3c) an amino acid sequence having 80% or more homology with the amino acid sequence of SEQ ID NO: 1.

7. The production method according to any one of 2 to 6, in which NDST2 and Hs3st1 are derived from a mouse or a human.
8. The production method according to any one of 2 to 7, in which the gene that encodes NDST2 comprises a base sequence that is any one of the following base sequences (1A) to (1D) and encodes an amino acid sequence of a protein having the function of NDST2:

(1A) the base sequence of SEQ ID NO: 4,

(1B) a base sequence in which one to several bases are deleted, substituted or added in the base sequence of SEQ ID NO: 4,

(1C) a base sequence which hybridizes under stringent conditions with a base sequence complementary to the base sequence of SEQ ID NO: 4, and

(1D) a base sequence having 80% or more homology with the base sequence of SEQ ID NO: 4.

9. The production method according to any one of 2 to 8, in which the gene that encodes Hs3st1 comprises a base sequence that is any one of the following base sequences (2A) to (2D) and encodes an amino acid sequence of a protein having a function of Hs3st1:

(2A) a base sequence of SEQ ID NO: 5,

(2B) a base sequence in which one to several bases are deleted, substituted or added in the base sequence of SEQ ID NO: 5,

(2C) a base sequence which hybridizes under stringent conditions with a base sequence complementary to the base sequence of SEQ ID NO: 5, and

(2D) a base sequence having 80% or more homology with the base sequence of SEQ ID NO: 5.

10. The production method according to any one of 1 to 9, in which the gene that encodes an extracellular domain of SDC is any one of the following base sequences (3A) to (3D) and comprises a base sequence that encodes an amino acid sequence of a protein having the function of the extracellular domain of SDC:

(3A) a base sequence of SEQ ID NO: 6,

(3B) a base sequence in which one to several amino acids are deleted, substituted or added in the base sequence of SEQ ID NO: 6,

(3C) a base sequence which hybridizes under stringent conditions with a base sequence complementary to the base sequence of SEQ ID NO: 6, and

(3D) a base sequence having 80% or more homology with the base sequence of SEQ ID NO: 6.

11. A recombinant cell in which a gene that encodes an extracellular domain of SDC is introduced into a mammalian cell that produces a heparin-like substance.
12. The recombinant cell according to 11, in which the mammalian cell that produces a heparin-like substance is a cell into which at least one of a gene that encodes NDST2 and a gene that encodes Hs3st1 is introduced.
13. The recombinant cell according to 11 or 12, in which the mammalian cell is a CHO cell.
14. A method for producing a cell that secretes and produces a heparin-like substance, the method comprising: introducing a recombinant expression vector comprising a gene that encodes an extracellular domain of SDC into a mammalian cell that produces a heparin-like substance.

### Effects of the Invention

According to the method for producing a heparin-like substance of the present invention, a heparin-like substance can be secreted and produced in a culture supernatant by introducing a gene that encodes an extracellular domain of syndecan into a mammalian cell that produces a heparin-like substance, and the heparin-like substance can be recovered from the culture supernatant. This solves the problem that a heparin-like substance as a product adheres to a cell membrane, and does not require a complicated purification process. Therefore, according to the production method of the present invention, a heparin-like substance can be efficiently produced without using an animal-derived tissue.

### Description of the Drawings

FIG. 1 is a diagram showing results of measuring an amount of sGAG in a culture supernatant of CHO/NH-SRGN cells and CHO/NH-SDC cells by an sGAG assay.

FIG. 2 is a diagram showing results of measuring the amount of sGAG in the culture supernatant of CHO/NH-SRGN cells and CHO/NH-SDC cells by HPLC.

FIGS. 3(A) and 3(B) are diagrams showing results of analyzing a sugar chain structure of heparin in the culture supernatant of CHO/NH-SRGN cells and CHO/NH-SDC cells using HPLC, in which FIG. 3(A) shows results of quantifying a concentration of a heparin-like sugar chain, and FIG. 3(B) shows results of quantifying a ratio of the heparin-like sugar chain in sulfation, respectively.

FIG. 4 is a graph showing results of measuring an amount of sGAG after serum-free acclimatization.

FIG. 5 is a graph showing results of calculating a production rate of sGAG after the serum-free acclimatization.

FIG. 6 shows results of measuring an expression level of a target protein by ELISA.

FIGS. 7(A) to 7(C) show results of measuring an expression level of an introduced gene by qRT-PCR, in which FIG. 7(A) shows an expression level of a NDST2 gene, FIG. 7(B) shows an expression level of an Hs3st1 gene, and FIG. 7(C) shows an expression level of an SDC gene.

FIG. 8 shows results of quantifying sGAG of a sample used for analysis of the sugar chain structure by HPLC.

FIG. 9 is a diagram showing chromatograms obtained from standard heparin-derived unsaturated disaccharides (HS-standards) and each sample by HPLC.

FIGS. 10(A) and 10(B) are diagrams showing results of analyzing the sugar chain structure of heparin in the culture supernatant of each cell using HPLC, in which FIG. 10(A) shows results of quantifying a concentration of a heparin-like sugar chain, and FIG. 10(B) shows results of quantifying a ratio of the heparin-like sugar chain in sulfation.

FIG. 11 shows a calibration curve used for evaluation of anticoagulation activity.

### EMBODIMENTS FOR CARRYING OUT THE INVENTION

Hereinafter, the terms used in the present specification have meanings generally used in the field unless otherwise specified.

### Method for Producing Heparin-Like Substance

A method for producing a heparin-like substance according to the present invention comprises the following steps (1) to (3):

(1) preparing a mammalian cell that produces a heparin-like substance;

(2) preparing a recombinant cell in which a gene that encodes an extracellular domain of syndecan is introduced into the mammalian cell that produces a heparin-like substance and is prepared in step (1); and

(3) culturing the recombinant cell prepared in step (2) in a medium, and collecting a heparin-like substance from a resulting culture supernatant.

Hereinafter, each step will be described.

### Step (1): Preparing a Mammalian Cell that Produces a Heparin-Like Substance

Step (1) is a step of preparing a mammalian cell that produces a heparin-like substance by introducing a gene that encodes a protein necessary for heparin biosynthesis into a mammalian cell serving as a host.

In the present invention, the “heparin-like substance” refers to a mixture of heparin and HS.

Examples of the mammalian cell include Chinese hamster-derived ovary CHO cells [Journal of Experimental Medicine, 108, 945 (1958); Proc. Natl. Acad. Sci. USA, 60, 1275 (1968); Genetics, 55, 513 (1968); Chromosoma, 41, 129 (1973); Methods in Cell Science, 18, 115 (1996); Radiation Research, 148, 260 (1997); Proc. Natl. Acad. Sci. USA, 77, 4216 (1980); Proc. Natl. Acad. Sci., 60, 1275 (1968); Cell, 6, 121 (1975); Molecular Cell Genetics, Appendix I, II (pp. 883-900)]; CHO cells lacking a dihydrofolate reductase gene (CHO/DG44 cells) [Proc. Natl. Acad. Sci. USA, 77, 4216 (1980)], CHO-K1 (ATCCL-61), DUkXB11 (ATCC CCL-9096), Pro-5(ATCC CCL-1781), and CHO-S (Life Technologies, Cat #11619) Pro-3, human umbilical vein endothelial cells (HUVEC), human umbilical artery endothelial cells (HUAEC), human lung microvascular vein endothelial cells (HLMVEC), human aortic endothelial cells (HAoEC), human coronary artery endothelial cells (HCAEC), human pulmonary artery endothelial cells (HPAEC), human embryonic kidney (HEK), rat myeloma cells YB2/3HL.P2.G11.16Ag.20 (also referred to as YB2/0”), monkey cells COS cells, mouse myeloma cells NSO, mouse myeloma cells SP2/0-Ag14, mouse myeloma cells BKK or HBT5637 (JP-A-563-000299). Among them, CHO cells, CHO/DG44 cells, or CHO-K1 (ATCC CCL-61) are preferable, and CHO cells are more preferable, from the viewpoint of production efficiency.

Examples of the protein required for heparin biosynthesis include bifunctional heparan sulfate N-deacetylase/N-sulfate transferase (hereinafter abbreviated as NDST2), heparan sulfate ester glucosamine 3-sulfate transferase 1 (hereinafter abbreviated as Hs3st1), heparan sulfate 2-O-sulfate transferase, C5-epimerase, and heparan sulfate 6-O-sulfate transferase. Among these, NDST2 and Hs3st1 are preferable. These proteins may be variant proteins as long as these proteins have activity required for heparin biosynthesis. These proteins are preferably derived from mammals, and more preferably derived from mice or humans.

Examples of the protein necessary for heparin biosynthesis include a protein that cannot be expressed in wild-type mammalian cells. Specifically, for example, when mammalian cells are CHO cells, CHO cells that produce a heparin-like substance can be obtained by introducing at least one of genes that encode NDST2 and Hs3st1, which are necessary for heparin biosynthesis and are not expressed in CHO cells (Bail J Y et al., Metab Eng 14: 81-90, 2012).

Examples of NDST2 include a protein comprising any one of the following amino acid sequences (1-a) to (1-c) and having a function of NDST2.

(1-a) Amino acid sequence of SEQ ID NO: 2 or amino acid sequence of NCBI accession NO: NP_003626.1

(1-b) Amino acid sequence in which one to several amino acids are deleted, substituted or added in amino acid sequence of SEQ ID NO: 2 or amino acid sequence of NCBI accession NO: NP_003626.1

(1-c) Amino acid sequence having 60% or more, preferably 80% or more, more preferably 90% or more, and most preferably 95% or more homology, with amino acid sequence of SEQ ID NO: 2 or amino acid sequence of NCBI accession NO: NP_003626.1

Examples of Hs3st1 include a protein comprising any one of the following amino acid sequences (2-a) to (2-c) and having a function of Hs3st1.

(2-a) Amino acid sequence of SEQ ID NO: 3 or amino acid sequence of NCBI accession NO: NP_034604.1

(2-b) Amino acid sequence in which one to several amino acids are deleted, substituted or added in amino acid sequence of SEQ ID NO: 3 or amino acid sequence of NCBI accession NO: NP_034604.1

(2-c) Amino acid sequence having 60% or more, preferably 80% or more, more preferably 90% or more, and most preferably 95% or more homology, with amino acid sequence of SEQ ID NO: 3 or amino acid sequence of NCBI accession NO: NP_034604.1

A polypeptide comprising an amino acid sequence in which one to several amino acids have been deleted, substituted or added in a target amino acid sequence can be obtained by introducing a site-specific mutation into DNA that encodes a polypeptide comprising the amino acid sequences of SEQ ID NO: 1 to 3, for example, by using a site-specific mutagenesis method [Molecular Cloning, A Laboratory Manual, Second Edition, Cold Spring Harbor Laboratory Press (1989), Current Protocols in Molecular Biology, John Wiley & Sons (1987-1997), Nucleic Acids Research, 10, 6487 (1982), Proc. Natl. Acad. Sci. USA, 79, 6409 (1982), Gene, 34, 315 (1985), Nucleic Acids Research, 13, 4431 (1985), Proc. Natl. Acad. Sci. USA, 82, 488 (1985)].

A range of “one to several” in “one to several amino acids are deleted, substituted or added” in the amino acid sequence is not particularly limited, but for example, when the number of amino acids in the amino acid sequence is 100 as one unit, “one to several” means 1, 2, 3, 4, 5, 6, 7, 8, 9, 10, 11, 12, 13, 14, 15, 16, 17, 18, 19, or 20, preferably about 1, 2. 3, 4, 5, 6. 7, 8, 9, or 10, and more preferably about 1, 2, 3, 4, or 5, per unit.

The term “amino acids are deleted” means loss or disappearance of an amino acid residue in the sequence, the term “amino acids are substituted” means that an amino acid residue in the sequence is replaced with another amino acid residue, and the term “amino acids are added” means that a new amino acid residue is added so as to be inserted into the sequence.

As a specific aspect of “one to several amino acids are deleted, substituted or added”, there is an aspect in which one to several amino acids are replaced with other chemically similar amino acids. Examples thereof include a case where a certain hydrophobic amino acid is substituted with another hydrophobic amino acid and a case where a certain polar amino acid is substituted with another polar amino acid having the same charge. Such chemically similar amino acids are known in the art for each amino acid.

Specific examples of a nonpolar (hydrophobic) amino acid include alanine, valine, glycine, isoleucine, leucine, proline, tryptophan, phenylalanine, and methionine. Examples of a polar (neutral) amino acid include serine, threonine, tyrosine, glutamine, asparagine, and cysteine. Examples of a basic amino acid having a positive charge include arginine, histidine, and lysine. Examples of an acidic amino acid having a negative charge include aspartic acid and glutamic acid.

Examples of the amino acid sequence having deletion, substitution, addition, or the like of one to several amino acids in the amino acid sequence of the target protein include an amino acid sequence having a certain sequence identity or more with the amino acid sequence of the target protein, and examples thereof include an amino acid sequence having 60% or more, preferably 65% or more, preferably 70% or more, preferably 75% or more, preferably 80% or more, preferably 85% or more, more preferably 90% or more, and still more preferably 95% or more sequence identity with the amino acid sequence of the target protein.

Examples of the gene that encodes NDST2 include a gene comprising a base sequence that is any one of the following base sequences (1-A) to (1-D) and encodes an amino acid sequence of a protein having a function of NDST2.

(1-A) Base sequence of SEQ ID NO: 4 or base sequence of NCBI accession NO: NM_003635.3

(1-B) Base sequence in which one to several bases are deleted, substituted or added in base sequence of SEQ ID NO: 4 or base sequence of NCBI accession NO: NM_003635.3

(1-C) Base sequence which hybridizes under stringent conditions with base sequence complementary to base sequence of SEQ ID NO: 4 or base sequence of NCBI accession NO: NM_003635.3

(1-D) Base sequence having at least 60% or more, preferably 80% or more, and more preferably 95% or more homology, with base sequence of SEQ ID NO: 4 or base sequence of NCBI accession NO: NM_003635.3

Examples of the gene that encodes Hs3st1 include a gene comprising a base sequence that is any one of the following base sequences (2-A) to (2-D) and encodes an amino acid sequence of a protein having a function of Hs3st1.

(2-A) Base sequence of SEQ ID NO: 5 or base sequence of NCBI accession NO: NM_010474.2

(2-B) Base sequence in which one to several bases are deleted, substituted or added in base sequence of SEQ ID NO: 5 or base sequence of NCBI accession NO: NM_010474.2

(2-C) Base sequence which hybridizes under stringent conditions with base sequence complementary to base sequence of SEQ ID NO: 5 or base sequence of NCBI accession NO: NM_010474.2

(2-D) Base sequence having at least 60% or more, preferably 80% or more, and more preferably 95% or more homology, with base sequence of SEQ ID NO: 5 or base sequence of NCBI accession NO: NM_010474.2

The base sequence which hybridizes under stringent conditions refers to a base sequence of hybridizable DNA obtained by a colony hybridization method, a plaque hybridization method, a Southern blot hybridization method, a DNA microarray method, or the like, using DNA comprising a target base sequence as a probe.

Specific examples thereof include a base sequence of DNA that can be identified by performing a hybridization method [Molecular Cloning, A Laboratory Manual, Second Edition, Cold Spring Harbor Laboratory Press (1989), Current Protocols in Molecular Biology, John Wiley & Sons (1987-1997), DNA Cloning 1: Core Techniques, A Practical Approach, Second Edition, Oxford University, (1995)] at 65° C. in the presence of 0.7 mol/L to 1.0 mol/L of sodium chloride using hybridized colony or plaque derived DNA, or a filter or slide glass on which a PCR product or oligo DNA having the sequence is immobilized, and then washing the filter or slide glass at 65° C. using an SSC solution (a composition of the SSC solution at a concentration of 1 time is composed of 150 mmol/L sodium chloride and 15 mmol/L sodium citrate) at a concentration of 0.1 to 2 times.

Examples of DNA comprising a base sequence which hybridizes under stringent conditions include DNA having a certain level or more of sequence identity with a base sequence of DNA having a base sequence of a gene to be used as a probe. For example, DNA having at least 60% or more, preferably 80% or more, and more preferably 95% or more homology, with the target base sequence is exemplified. For example, when the number of bases in the base sequence is 100 as one unit, DNA is exemplified comprising a base sequence having deletion, substitution, addition, or the like of one to several bases, preferably 1 to 40 bases, preferably 1 to 35 bases, preferably 1 to 30 bases, preferably 1 to 25 bases, preferably 1 to 20 bases, more preferably 1 to 15 bases, still more preferably 1, 2, 3, 4, 5, 6, 7, 8, 9, or 10 bases, and yet still more preferably 1, 2, 3, 4, or 5 bases, per unit in the base sequence of the target gene.

The term “deletion of a base” means that the base in the sequence is lost or disappeared, the term “substitution of a base” means that the base in the sequence is replaced with another base, and the term “addition of a base” means that a new base is added so as to be inserted.

The base sequence of a gene that encodes a protein of a eukaryote often shows polymorphism of a gene. The gene used in the present invention also contains a gene having a small mutation in the base sequence due to such a polymorphism.

A numerical value of homology in the present invention may be a numerical value calculated using a homology search program known to those skilled in the art, unless otherwise specified, and examples of the numerical value of homology in the present invention include a numerical value calculated using default parameters in BLAST [J. Mol. Biol., 215, 403 (1990)] for the base sequence, and a numerical value calculated using default parameters in BLAST 2 [Nucleic Acids Res., 25, 3389 (1997),Genome Res., 7, 649 (1997), https://blast.ncbi.nlm.nih.gov/Blast.cgi?PAGE_TYPE=BlastSearch&BLAST_SPEC=blast 2seq&LINK_LOC=align2seq] for the amino acid sequence.

The default parameters are: G (Cost to open gap) is 5 for a base sequence and 11 for an amino acid sequence, -E (Cost to extend gap) is 2 for a base sequence and 1 for an amino acid sequence, -q (Penalty for nucleotide mismatch) is −3, -r (reward for nucleotide match) is 1, -e (expect value) is 10, -W (wordsize) is 11 residues for a base sequence and 3 residues for an amino acid sequence, -y [Dropoff(X) for blast extensions in bits] is 20 for blastn, 7 for non-blastn programs, -X (X dropoff value for gapped alignment in bits) is 15 and -Z (final X dropoff value for gapped alignment in bits) is 50 for blastn and 25 for non-blastn programs (https://blast.ncbi.nlm.nih.gov/Blastcgi?PAGE_TYPE=BlastSearch).

The mammalian cell that produces a heparin-like substance can be obtained by introducing a recombinant vector comprising cDNA that encodes a full length or partial length of a protein required for heparin biosynthesis into a mammalian cell serving as a host cell.

As the recombinant vector, any vector can be used as long as the vector can be autonomously replicated or incorporated into a chromosome in a host cell to be used and contains an appropriate promoter at a position where DNA that encodes a polypeptide can be transcribed.

Although a transcription termination sequence is not necessarily required for the recombinant vector, it is preferable to arrange the transcription termination sequence immediately below a structural gene. Furthermore, the recombinant vector may contain a gene that controls a promoter.

As the recombinant vector, it is preferable to use a plasmid in which a Kozak sequence, which is a ribosomal binding sequence, is appropriately arranged around a start codon.

In a base sequence of DNA, the base can be substituted so as to be an optimal codon for expression in the host, thereby improving a production rate of the target NDST2 and Hs3st1.

As the recombinant vector, any vector can be used as long as the vector can exhibit functions in animal cells, and examples thereof include pcDNA I, pcDM8 (Funakoshi Corp.), pAGE107 [JP-A-03-22979; Cytotechnology, 3, 133 (1990)], pAS3-3 (JP-A-02-227075), pcDM8 [Nature, 329, 840 (1987)), pcDNA I/Amp (Invitrogen Corp.], pcDNA3.1 (Invitrogen Corp.), pREP4 (Invitrogen Corp.), pAGE103 [J. Biochemistry, 101, 1307 (1987)], pAGE210, pME18SFL3, pKANTEX93 (WO 97/10354), N5KG1va1 (specification of U.S. Pat. No. 6,001,358), INPEP4 (Biogen-IDEC Corp.), and a transposon vector (WO 2010/143698).

As the promoter, any promoter can be used as long as the promoter can exhibit functions in animal cells, and examples thereof include a promoter of an immediate early (IE) gene of cytomegalovirus (CMV), a SV40 early promoter, a retrovirus promoter, a metallothionein promoter, a heat shock promoter, an SRα promoter, and a promoter or enhancer of Moloney murine leukemia virus. An enhancer of the IE gene of human CMV may be used together with a promoter.

The recombinant vector may comprise a selection marker. The “selection marker” is a gene that enables selection of cells comprising the gene. The term “positive selection” refers to a process by which a positive selection occurs and a cell comprising a selection marker is selected. An example of the selection marker with positive drug resistance is that a cell comprising the marker survives in a drug-containing culture medium, and a cell without the marker is killed. Examples of the selection marker include a drug resistance gene such as neo giving G418 resistance; hygr giving hygromycin resistance; and puro giving puromycin resistance. Other positive selection marker genes include a gene that enables identification or screening of cells comprising the marker. Examples of these genes include a fluorescent protein (GFP and GFP-like chromophore, luciferase) gene, a lacZ gene, an alkaline phosphatase gene, and a surface marker such as CD8. The term “negative selection” refers to a process by which a cell comprising a negative selection marker is killed by being exposed to an appropriate negative selection drug. For example, a cell comprising a simple herpesvirus kinase (HSV-tk) gene [Wigler et al, Cell 11: 223 (1977)] is sensitive to a drug gamma cyclobilla (GANC). Similarly, a gpt gene makes cells sensitive to 6-thioxanthine.

As a method for introducing a recombinant vector into a host cell, any method for introducing DNA into an animal cell can be used. Examples thereof include an electroporation method [Cytotechnology, 3, 133 (1990)], a calcium phosphate method (JP-A-02-227075), and a lipofection method [Proc. Natl. Acad. Sci. USA, 84, 7413 (1987)].

### Step (2): Preparing a Recombinant Cell in Which a Gene that Encodes an Extracellular Domain of Syndecan is Introduced Into the Mammalian Cell that Produces a Heparin-Like Substance and is Prepared in Step (1)

Step (2) is a step of preparing a recombinant cell by introducing a recombinant vector comprising a gene that encodes an extracellular domain of syndecan into the mammalian cell that produces a heparin-like substance and prepared in step (1).

A structure of syndecan comprises an extracellular domain, a transmembrane domain, and a cytoplasmic domain. Among them, the extracellular domain of syndecan comprises a glycosaminoglycan binding site. It is preferable that syndecan has at least a glycosaminoglycan binding site as an extracellular domain. The extracellular domain of syndecan is preferably derived from a mammal, and more preferably derived from a mouse or a human.

Examples of the extracellular domain of syndecan include a protein comprising any one of the following amino acid sequences (3-a) to (3-c) and having a function of the extracellular domain of syndecan.

(3-a) Amino acid sequence of SEQ ID NO: 1 or amino acid sequence of NCBI accession NO: NP_001006947.1

(3-b) Amino acid sequence in which one to several amino acids are deleted, substituted or added in amino acid sequence of SEQ ID NO: 1

(3-c) An amino acid sequence having 60% or more, preferably 80% or more, more preferably 90% or more, and most preferably 95% or more homology, with amino acid sequence of SEQ ID NO: 1

The amino acid sequence of SEQ ID NO: 1 is an amino acid sequence at positions 1 to 229 from the N-terminal of the amino acid sequence of NCBI accession number NP_001006947.1 (total length 310 amino acids).

Examples of the gene that encodes the extracellular domain of syndecan include a gene comprising a base sequence that is any one of the following base sequences (3-A) to (3-D) and encodes an amino acid sequence of a protein having the function of an extracellular domain of syndecan.

(3-A) Base sequence of SEQ ID NO: 6 or base sequence of NCBI accession NO: NM_001006946.1

(3-B) Base sequence in which one to several bases are deleted, substituted or added in base sequence of SEQ ID NO: 6 or base sequence of NCBI accession NO: NM_001006946.1

(3-C) Base sequence which hybridizes under stringent conditions with base sequence complementary to base sequence of SEQ ID NO: 6 or base sequence of NCBI accession NO: NM_001006946.1

(3-D) Base sequence having at least 60% or more, preferably 80% or more, and more preferably 95% or more homology, with base sequence of SEQ ID NO: 6 or base sequence of NCBI accession NO: NM_001006946.1

In step (2), a recombinant cell that can secrete a heparin-like substance into a culture supernatant is obtained by introducing the recombinant vector comprising the gene that encodes the extracellular domain of syndecan, that is, cDNA that encodes a whole length or partial length of the extracellular domain of syndecan into the mammalian cell that produces a heparin-like substance and is prepared in step (1).

A method for introducing a recombinant vector and a recombinant vector into a host cell is the same as in step (1).

### (3) Culturing the Recombinant Cell Prepared in Step (2) in a Medium, and Collecting a Heparin-Like Substance from a Resulting Culture Supernatant

Step (3) is a step of culturing the recombinant cell prepared in step (2) in a medium to secrete and produce a heparin-like substance in the medium by the recombinant cell, and recovering the heparin-like substance from the resulting culture supernatant.

In step (3), the recombinant cell is preferably cultured under conditions for promoting the production of the heparin-like substance. Specifically, for example, it is preferable to culture the recombinant cell in a medium that enables production of the heparin-like substance in the recombinant cell and promotes secretion of the heparin-like substance from the recombinant cell into the culture supernatant.

The medium preferably contains carbon, nitrogen, oxygen, and other nutrients, a growth factor, a buffer, a cofactor, and other optional substances sufficient to maintain at least viability of cells and enable expression of heparin-like substances. In the embodiment in which the gene that encodes a heparin-like substance is under control of an inducible promoter or contains an inducible promoter, the medium may further contain an inducer.

Examples of the medium include RPMI or DMEM to which 10% fetal bovine serum (FCS) is added, and a tissue culture medium to which a factor such as an antimicrobial agent, a growth factor, and other cytokines is added (for example, Cell Biology (Third Edition) A Laboratory Handbook, vol. 1, 2006, Elsevier Inc.). Specific examples thereof include a medium preparation known to those skilled in the art, such as RPMI, IMDM, DMEM, DMEM/F12, and serum-free or low-serum EMEM. These media may contain an additional nutritional supplement food such as antibiotics, lipids, transferrin, insulin, and amino acids, and as necessary, a cofactor.

From the viewpoint of promoting the production of the heparin-like substance, the medium preferably contains at least one selected from glucose, sulfates, and phosphoric acid. A concentration of the glucose in the medium is usually preferably 5 mM to 75 mM, more preferably 10 mM to 60 mM, and still more preferably 15 mM to 35 mM. A concentration of the sulfate in the medium is usually preferably 0.5 mM to 50 mM, more preferably 10 mM to 50 mM, and still more preferably 30 mM to 50 mM. A concentration of the phosphate in the medium is usually preferably 0.5 mM to 50 mM, more preferably 1 mM to 50 mM, and still more preferably 10 mM to 50 mM.

The heparin-like substance can be produced by producing and accumulating the heparin-like substance in the culture supernatant and collecting the heparin-like substance from the culture supernatant. A method of culturing a recombinant cell in a medium can be performed according to a usual method. Culture is usually performed for 1 day to 7 days under conditions such as pH 6 to 8, 30° C. to 40° C., and 5% CO₂.

The secretion and production of the heparin-like substance from the recombinant cell can be confirmed by adding an enzyme solution containing heparin-lyase I, II, or III to a culture supernatant, subjecting the culture supernatant to an enzyme treatment, and then performing quantitative determination by HPLC for unsaturated disaccharide analysis, as will be described later in Examples. Specifically, it is assumed that the heparin-like substance is secreted and produced from the recombinant cell into the culture supernatant when an amount of at least one selected from 2SNS6 in which an amino group of glucosamine and three sulfate groups are bonded at 6-position, 2SNS in which a sulfate group is bonded to 2-position glucuronic acid and a glucosamine amino group, NS6S in which an amino group of glucosamine and two sulfates are bonded at 6-position, and NS in which a sulfate group is bonded to glucosamine N is increased in the culture supernatant of the recombinant cell as compared with the culture supernatant of a control (wild-type) cell. The secretion production of the heparin-like substance from the recombinant cell can also be confirmed by measuring an amount of sGAG (sulfated GAG) in the culture supernatant, as will be described later in Examples.

In step (3), proteoglycan, which is a glycoprotein comprising a core protein bound to a heparin-like substance, which is GAG, is secreted from the recombinant cell into the culture supernatant. Isolation of proteins from the culture supernatant is performed by a common method known in the art. For example, a tag that facilitates isolation of the heparin-like substance, such as an affinity tag, may be used. Examples of the tag include polyhistidine (His6 tag), a nickel matrix, a chitin binding protein (CBP), a maltose binding protein (MBP), glutathione-S-transferase (GST), a FLAG tag, and an epitope tag.

Isolation of the heparin-like substance from the core protein is performed by a method known in the art. Examples thereof include an enzyme digestion method using heparinase, and a treatment with sodium hydroxide or alkaline boron hydride.

The heparin-like substance to be isolated preferably contains 50% or more, more preferably 60% or more, still more preferably 70% or more, particularly preferably 80% or more of a sulfated disaccharide.

The heparin-like substance to be isolated includes, for example, the following (1) to (20).

(1) 10% to 50% of trisulfated disaccharide, 30% to 50% of disulfated disaccharide, and 10% to 30% of monosulfated disaccharide.
(2) 10% to 40% of trisulfated disaccharide, 35% to 45% of disulfated disaccharide, and 10% to 25% of monosulfated disaccharide.
(3) 15% to 40% of trisulfated disaccharide, 35% to 45% of disulfated disaccharide, and 10% to 25% of monosulfated disaccharide.
(4) 5% to 25% of trisulfated disaccharide, 30% to 50% of disulfated disaccharide, and 10% to 20% of monosulfated disaccharide.
(5) 1% to 30% of trisulfated disaccharide, 25% to 55% of disulfated disaccharide, and 5% to 40% of monosulfated disaccharide.
(6) 15% to 40% of trisulfated disaccharide.
(7) 35% to 45% of disulfated disaccharide.
(8) 10% to 25% of monosulfated disaccharide.
(9) 10% to 50% of trisulfated disaccharide.
(10) 30% to 50% of disulfated disaccharide.
(11) 10% to 30% of monosulfated disaccharide.
(12) 15% to 30% of trisulfated disaccharide.
(13) 40% to 45% of disulfated disaccharide.
(14) 10% to 15% of monosulfated disaccharide.
(15) 5% to 25% of trisulfated disaccharide.
(16)1% to 30% of trisulfated disaccharide.
(17) 30% to 50% of disulfated disaccharide.
(18) 25% to 55% of disulfated disaccharide.
(19) 10% to 20% of monosulfated disaccharide.
(20) 5% to 40% of monosulfated disaccharide.

The heparin-like substance may contain a repeating structure of a disaccharide unit having various lengths. The heparin-like substance preferably contains any of UA-GlcNAc(6S), UA(2S)-GlcNAc, UA-(2S)-GlcNAc(6S), UA-GlcNS, UA-GlcNS(6S), UA(2S)-GlcNS, and UA(2S)-GlcNS(6S), and these may be present in any order in the heparin-like substance.

In the above, UA is a uronic acid residue (that is, glucuronic acid or iduronic acid), Ac is acetyl, GlcNAc is N-acetyl glucosamine, GlcNS is glucosamine-N-sulfate, 2S is 2-O-sulfonate, and 6S is 6-O-sulfate.

### Recombinant Cell

An aspect of the present invention is a recombinant cell in which a gene that encodes an extracellular domain of SDC is introduced into the mammalian cell that produces a heparin-like substance. The recombinant cell can be prepared by preparing a mammalian cell that produces a heparin-like substance and introducing a gene that encodes an extracellular domain of syndecan into the mammalian cell in the same manner as described above in step (1) and step (2) of <Method for producing heparin-like substance>.

### Method for Producing Cell that Secretes and Produces Heparin-Like Substance

An aspect of the present invention is a method for producing a cell that secretes and produces a heparin-like substance. The method comprises: introducing a recombinant expression vector comprising a gene that encodes an extracellular domain of SDC into the mammalian cell that produces a heparin-like substance. The production method can be carried out by preparing a mammalian cell that produces a heparin-like substance and introducing a gene that encodes an extracellular domain of syndecan into the mammalian cell in the same manner as described above in steps (1) and (2) of <Method for producing heparin-like substance>.

### Pharmaceutical Composition

An aspect of the present invention is a pharmaceutical composition comprising a heparin-like substance or a fragment thereof produced by the method described in the present specification. In the present embodiment, the heparin-like substance or the fragment thereof may bind to the core protein. The pharmaceutical composition may contain other therapeutic agents. The pharmaceutical composition can be formulated by using, for example, a common vehicle or diluent, and a pharmaceutical additive (for example, an excipient, a binder, or a preservative) of a type suitable for a desired administration method.

Examples of a form of the pharmaceutical composition include an aqueous agent for sterile injection. The aqueous agent for sterile injection can be formulated according to a known technique by using an appropriate dispersant or wetting agent and suspending agent. The aqueous agent for sterile injection may be, for example, a nontoxic parenterally acceptable diluent such as a solution in 1,3-butanediol, or a solution or suspension for sterile injection in a solvent. The vehicle and the solvent that can be used are water, a Ringer's solution, and an isotonic saline solution.

A dosage form of the pharmaceutical composition is not particularly limited, and various dosage forms can be used. Examples of the dosage form for administering pharmaceutical compositions includes the composition being administered in a form of a tablet, a capsule, a sachet, a troche, a pill, a powder, a granule, an elixer, a tincture, a solution, a suspending agent, an elixer, a syrup, an ointment, a cream, or the like, intravenous administration or injection, a paste, an emulsion or a solution. In addition, examples thereof include transdermal administration by a patch mechanism or an ointment. Any of these may be modified into a sustained release preparation and/or a continuous release preparation.

Examples of a pharmaceutically acceptable carrier include, but are not limited to, a vehicle, an adjuvant, a surfactant, a suspending agent, an emulsifier, an inert filler, a diluent, an excipient, a wetting agent, a binder, a lubricant, a buffer, a disintegrating agent, and a carrier. Typically, the pharmaceutically acceptable carrier is chemically inactive to an active compound, and does not have harmful side effects or toxicity under use conditions. Properties of the pharmaceutically acceptable carrier may vary depending on other features of the specific dosage form and the composition to be used.

### Treatment Method and Preventing Method

An aspect of the present invention includes a method for treating or preventing blood coagulation or a state associated with or caused by blood coagulation in a subject requiring a heparin-like substance or a fragment thereof. The method comprises administering the heparin-like substance or the fragment thereof produced by the method described in the present specification. In the present embodiment, the heparin-like substance or the fragment thereof may bind to the core protein. Examples of the state associated with or caused by blood coagulation include acute coronary syndrome, atrial fibrillation, deep venous thrombosis, and pulmonary embolization.

The subject refers to a mammal. Examples of the mammal include a human, a primate, a livestock animal (for example, sheep, bovine, horse, donkey, pig), a companion animal (for example, dog, cat), a laboratory test animal (for example, mouse, rabbit, rat, guinea pig, hamster), and a capture wild animal (for example, rice, sika). The mammal is typically a human or a primate, and more typically a human.

It is preferable that a dose and dosing time is a dose and time at which a therapeutic profit is provided in treatment, prevention, or management of blood coagulation or a state associated with blood coagulation. A specific effective dose and dosing time may vary depending on a factor such as a state, a medical history, a physique, a body weight, and an age of the subject.

### EXAMPLE

Examples will be described below, but the present invention is not limited to the following Examples.

### 1. Evaluation of Heparin-Producing Ability in CHO/NH-SRGN Cells and CHO/NH-SDC Cells

### Preparation of CHO/NH-SRGN Cells and CHO/NH-SDC Cells

Based on Bail J Y et al., Metab Eng14:81-90, 2012, DNA that encodes NDST2 (base sequence represented by SEQ ID NO: 4) and DNA that encodes Hs3st1 (base sequence represented by SEQ ID NO: 5) were co-introduced to obtain a CHO/NH cell clone #3 (CHO-NH #3 cell).

A vector PB513B-1_SRGN-IRES-EGFP used to prepare CHO/NH-SRGN cells was based on PiggyBac Transposon Vector PB513B-1, and was a vector having an EF1α promoter of a Chinese hamster, an IRES element, and a mycHis tag sequence at C-terminus. In the vector PB513B-1_SRGN-IRES-EGFP, DNA that encodes hSRGN (base sequence represented by SEQ ID NO: 7) was introduced under the control of the EF1α promoter so as to be co-expressed with an EGFP gene and a puromycin resistance gene, which are screening markers.

CHO/NH-SRGN cells were prepared by introducing DNA that encodes human sericin glycine (SRGN) (base sequence represented by SEQ ID NO: 7) into CHO/NH #3 cells using a vector PB513B-1_SRGN-IRS-EGFP, and CHO/NH-SDC cells were prepared by introducing DNA of an extracellular domain of human syndecan (SDC) (base sequence represented by SEQ ID NO: 6) into CHO/NH #3 cells using a vector PB513B-1_SDC-IRS-EGFP.

A vector PB513B-1_SDC-IRES-EGFP used to prepare CHO/NH-SDC cells was based on PiggyBac Transposon Vector PB513B-1, and was a vector having an EF1α promoter of a Chinese hamster, an IRES element, and a mycHis tag sequence at C-terminus. In the vector PB513B-1_SDC-IRES-EGFP, DNA that encodes SRGN (base sequence represented by SEQ ID NO: 7) was introduced under the control of the EF1α promoter so as to be co-expressed with an EGFP gene and a puromycin resistance gene, which are screening markers.

The culture supernatant of each cell prepared as described above was analyzed to evaluate heparin-producing ability.

### Measurement of Amount of sGAG in Culture Supernatant of CHO/NH-SRGN Cells and CHO/NH-SDC Cells

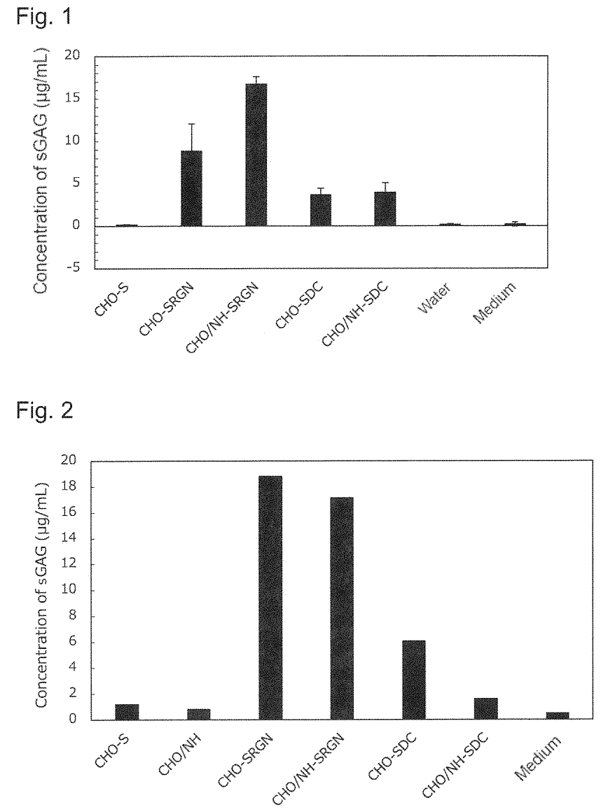

Each cell was cultured in a good proliferation state, and then seeded in a 6-well plate at 0.6×10⁶ cells/well, and a culture supernatant collected after 3 days without medium change was used as a sample to measure an amount of sGAG by an sGAG assay. The sGAG assay is an assay that colorimetrically quantifies a total amount of sGAG in a solution using a dye (1,9-dimethylmethylene blue) that specifically binds to a sulfated sugar chain. The results are shown in FIG. 1.

### Analysis Results of Sugar Chain Structure by HPLC

Since the production of heparin in the culture supernatant was suggested by the sGAG assay, the sugar chain structure of the same sample was analyzed by HPLC. The sGAG quantification results of the samples used for the analysis are shown in FIG. 2. Since a concentration of a CHO/NH-SDC cell sample was too low to confirm a presence of sufficient sugar chains for analysis, a spin column enrichment operation was performed.

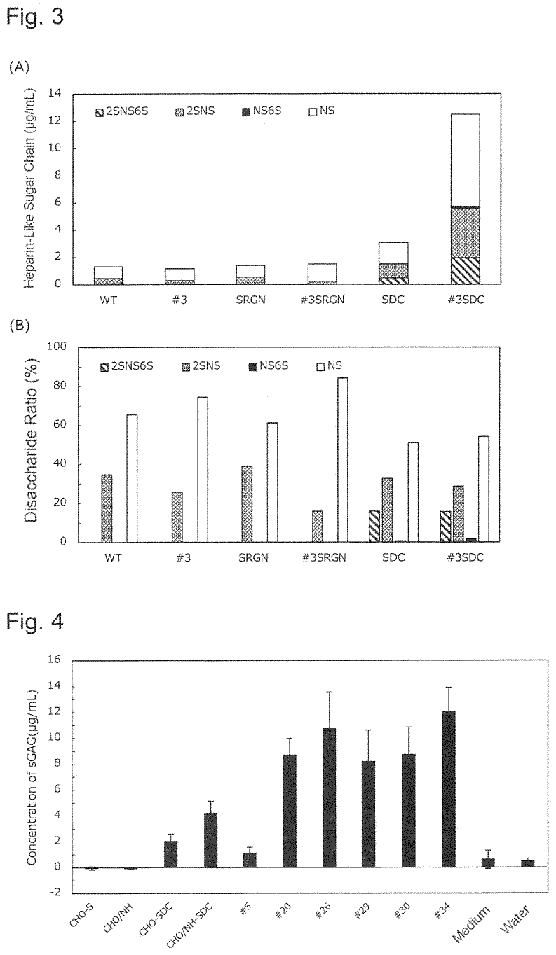

To confirm the production of heparin, first, 20 μL of a sample solution was directly taken, 10 μL of a buffer and 10 μL of an enzyme solution containing 10 mU of heparin lyase I, II or III were added thereto, the mixture was enzymatically treated at 37° C. for 3 hours, after completion of the reaction, the enzyme was deactivated at 100° C. for 5 minutes, 20 μL of water was added after freeze-drying, and the amount was determined by HPLC for unsaturated disaccharide analysis. The results are shown in FIGS. 3A and 3B.

In FIGS. 3(A) and 3(B), WT represents a wild-type CHO cell, #3 represents a CHO-NH #3 cell, SRGN represents a CHO-SRGN cell prepared by introducing DNA that encodes SRGN (base sequence represented by SEQ ID NO: 7) into a CHO cell using a vector PB513B-1_SRGN-IRES-EGFP, #3 SRGN represents a CHO/NH-SRGN cell, and SDC represents a CHO-SDC cell obtained by introducing DNA of an extracellular domain of SDC (base sequence represented by SEQ ID NO: 6) into a CHO cell using a vector PBS 13B-1_SDC-IRES-EGFP.

As shown in FIGS. 3(A) and 3(B), in the culture supernatant of the SRGN-introduced cells (SRGN, #3 SRGN), no increase in the detection of heparin-like substances was detected as compared with WT or #3. On the other hand, in the culture supernatant of the SDC-introduced cells (SDC, #3 SDC), the production of heparin-like substances was increased as compared with WT or #3. From the above results, the CHO/NH-SDC cells were considered to be cells more suitable for producing heparin-like substances, and cloning and analysis of each clone were performed.

### 2. Measurement Results of Amount of GAG After Serum-Free Acclimatization

Since a sugar chain structure characteristic of heparin was observed, cloning of CHO/NH-SDC cells and analysis of each clone were carried out.

### Cloning and Serum-Free Acclimatization

CHO/NH-SDC cells were cultured using a serum-containing medium to be in an adhered state, and cloned in a collagen-coated 96-well plate by a limiting dilution method. At this time, for the culture from one cell, the culture was performed without adding a drug in consideration of the toxicity to the cell. Cells were scaled up with a collagen-coated 24- and 6-well plate, a plurality of clones were cultured, and 36 clones were established. Thereafter, each cell was seeded in a normal 6-well plate at 0.6×10⁶ cells/well, and the amount of sGAG in the culture supernatant was measured using, as a sample, a culture supernatant collected three days without medium exchange, and an upper 6 clones (#5, #20, #26, #29, #30, and #34) were subjected to serum-free acclimatization.

### Measurement of Amount of sGAG, Production Rate of sGAG, and Amount of SDC After Serum-Free Acclimatization

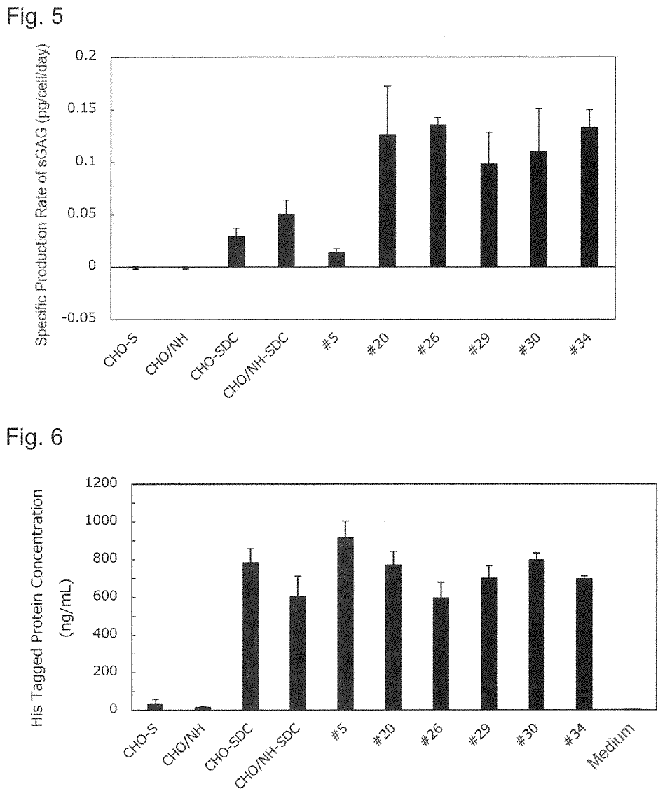

After the serum-free acclimatization, each cell was seeded on a normal 6-well plate at 0.6×10⁶ cells/well, and the amount of sGAG in the culture supernatant was measured using, as a sample, the culture supernatant collected 3 days without medium exchange. The results are shown in FIG. 4. The results of calculating the production rate of sGAG are shown in FIG. 5, and the results of quantifying the amount of SDC in the culture supernatant by ELISA are shown in FIG. 6.

As shown in FIG. 6, the evaluated 6 clones had the secretory production ability of SDC equal to or higher than that of CHO/NH-SDC cells, and #5 was 1.5 times or more that of CHO/NH-SDC cells, showing the highest expression level.

### Analysis of Expression Level of Introduced Gene by qRT-PCR

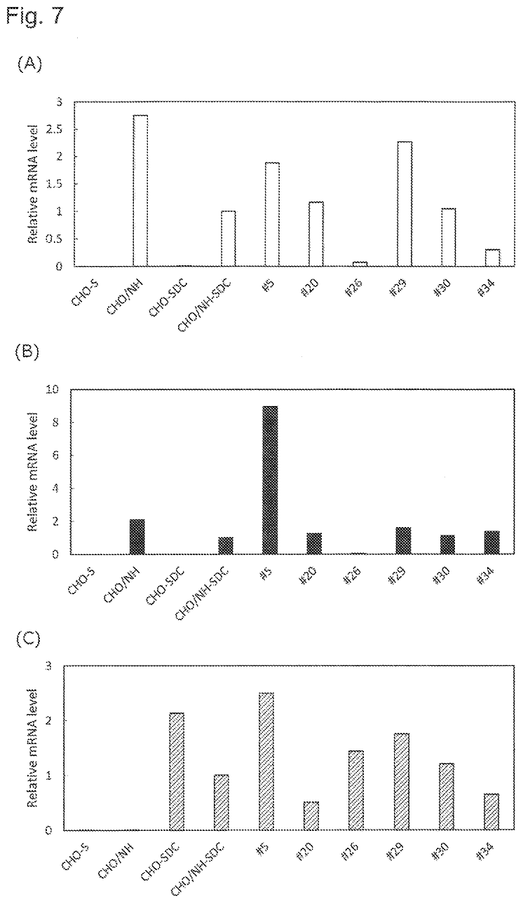

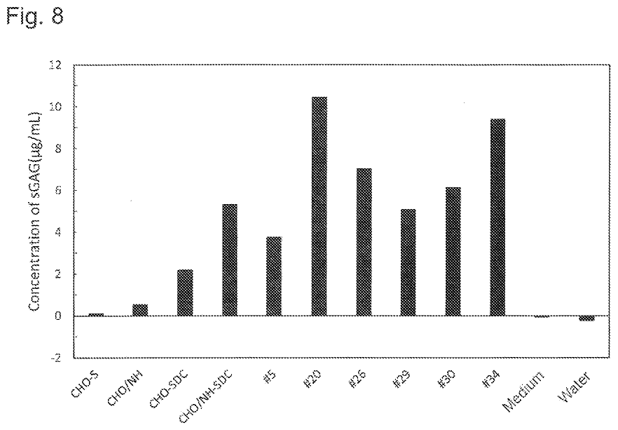

Each cell was seeded in a 6-well plate at 0.6×10⁶ cells/well, RNA was extracted and cDNA was prepared from cells cultured for 3 days without medium exchange, and expression levels of introduced genes (NDST2, Hs3st1 and SDC) were quantified by qRT-PCR. FIG. 7(A) shows an expression level of a NDST2 gene, FIG. 7(B) shows an expression level of an Hs3st1 gene, and FIG. 7(C) shows an expression levels of an SDC gene.

As shown in FIGS. 7(A) to 7(C), a high expression level of each gene was confirmed in #5, whereas no expression was observed in #26. #26 also showed low anticoagulant activity compared to other clones, suggesting that ATIII recognition-specific sequences are formed by NDST2 and Hs3st1.

### 3. Analysis of Sugar Chain Structure by HPLC

Sugar chain structures of heparin-like substances produced by various CHO cells were analyzed by HPLC.

### Analysis Conditions

As a pretreatment of a sample, 400 μL of culture supernatant of various CHO cells was centrifuged at 10000 rpm for 10 minutes, and 400 μL of two-stage distilled water (DDW) was added to an upper layer, followed by freeze-drying. Thereafter, 20 μL of the sample was separated from 40 μL of sample, subjected to Heparin lyase I, II and III treatments, 80 μL of DDW was added thereto, and 20 μL of the resultant was subjected to HPLC.

The treatment with a specific degrading enzyme was as follows. 20 82 L of an acetate buffer [0.1 MCH₃COONa, 10 mM (CH3COO)₂Ca (pH7.0)], 5 μL of 0.5 mU/μL Heparinase, 5 μL of 0.5 mU/μL Heparinase I, and 5 μL of 0.5 mU/μL Heparinase II were simultaneously added, and the mixture was incubated at 37° C. for 16 hours to decompose heparin-like sugar chains into unsaturated disaccharides. The mixture was heated in a boiling water bath for 3 minutes to deactivate the enzyme, lyophilized, dissolved in water, and subjected to an unsaturated disaccharide analysis by HPLC.

### Disaccharide Composition Analysis of HS and CS

A disaccharide composition analysis of HS and CS was performed using a device and analysis software shown below.

Pump of eluent: LIQUID CHROMATOGRAPHY LC-10Ai manufactured by Shimadzu Corporation

Detector: Intelligent Fluorescence Detector FP-920S manufactured by JASCO

Pump for post reaction: MLNICHEMI PUMP NP-FX(II)-1U manufactured by Nihon Seimitsu Kagaku Co., Ltd.

Integrator: Chromato Integrator D-2500 manufactured by Hitachi, Ltd.

Injector: sample injector (model 7725) manufactured by Reodyne Corporation

Column: Senshu Pak DOCOSIL(4.6 mm i.d.×150 mm) manufactured by Senshu Scientific Co., Ltd.

Column oven: L-7300 manufactured by Hitachi, Ltd.

Dry reaction tank: Dry Reaction Bath DB-5 manufactured by Takashima Keiki, Ltd.

Analysis software: Chromatocon-PRO manufactured by Runtime Instruments Co., Ltd.

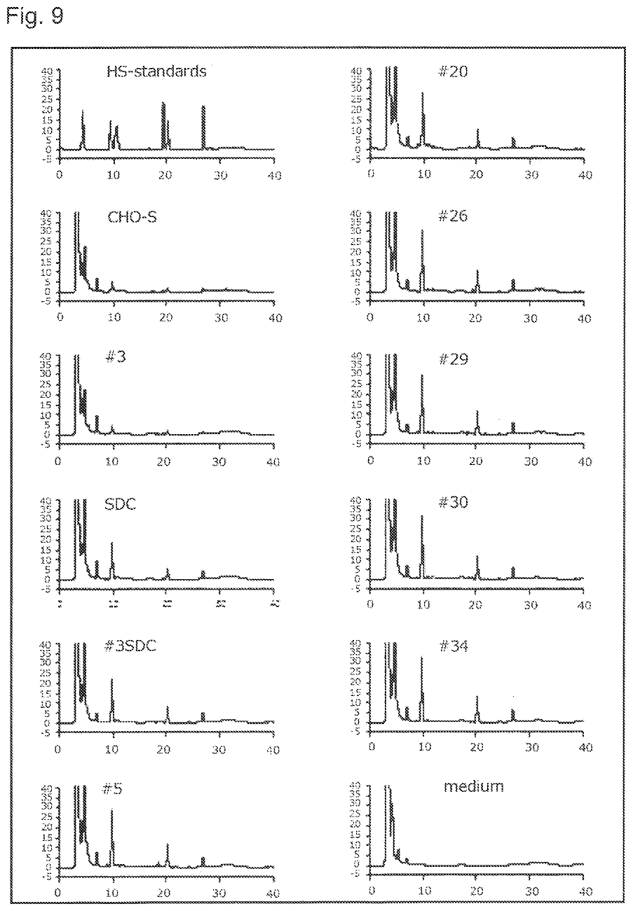

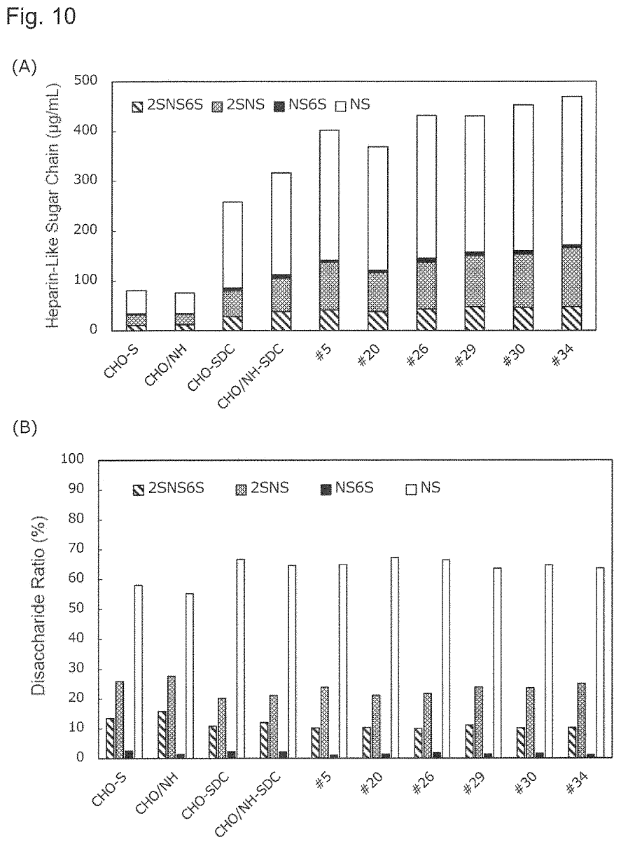

The results of quantifying sGAG in the sample used for the analysis by HPLC are shown in FIG. 8, and chromatograms obtained from standard heparin-derived unsaturated disaccharides (HS-Standards) and the sample are shown in FIG. 9. The results of quantifying a content of heparin-like sugar chains and a ratio of each sugar chain in sulfation by HPLC are shown in FIGS. 10(A) and 10(B), respectively. Table 1 shows a detected concentration of a sulfated sugar chain (HS) and a ratio of the sulfated sugar chain in the heparin-like substance.

TABLE 1

| | Concentration | Ratio |
|---|---|---|
| Sample | (μg/mL) | (%) |
| CHO-S | 81 | 23.1 |
| #3 | 76 | 23.3 |
| SDC | 258 | 21.6 |
| #3 SDC | 316 | 22.6 |
| #5 | 402 | 20.1 |
| #20 | 369 | 20.4 |
| #26 | 432 | 23.9 |
| #29 | 431 | 22.8 |
| #30 | 453 | 23.2 |
| #34 | 470 | 24.7 |

As shown in Table 1, a degree of sulfation of a sugar chain detected from samples obtained from the prepared clones (#5, #20, #26, #29, #30, and #34) was about the same as that of heparin, and the production of heparin-like sugar chain was significantly increased as compared with the control (CHO-S, SDC).

### Measurement of Anti-Factor Xa Activity

Measurement was performed using a test team heparin S measurement kit manufactured by Sekisui Medical Co., Ltd.

(http://www.info.pmda.go.jp/tgo/pack/13A2X00197218081_A_01_10/). The standard heparin was prepared using medical heparin (101 IU/mg). An outline of the measurement principle is as follows.

### Constituent Reagent Name and Component

1. Substrate: N-benzoyl-L-isoleucyl-L-glutamyl (γ-OR)-glycyl-L-arginyl-p-nitroanilide hydrochloride (S-2222)

2. Antithrombin III agent: Antithrombin III (derived from human)

3. Factor Xa agent: Factor Xa (derived from bovine)

4. Buffer: 2-amino-2-hydroxymethyl-1,3-propanediol buffer

5. Normal plasma agent: human normal plasma

### Verification of Anticoagulation Ability

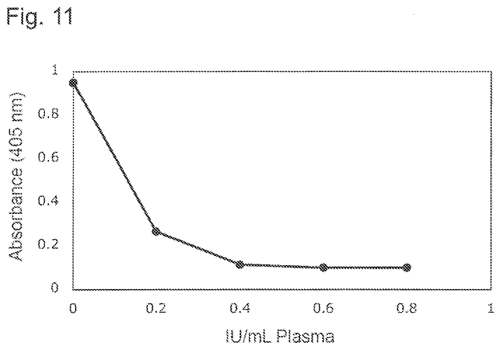

The anticoagulation activity (anti-FXa activity 9) of clones (#20, #26, #29, #30, and #34) having a high degree of sulfation in the above HPLC results was examined using a calibration curve shown in FIG. 11. The results are shown in Table 2.

TABLE 2

| | Anti-FXa |
|---|---|
| | activity |
| Sample | (IU/mL) |
| #20 | 0.09 |
| #26 | 0.02 |
| #29 | 0.1 |
| #30 | 0.09 |
| #34 | 0.09 |

As shown in Table 2, in samples obtained from #20, #29, #30 and #34, the anti-FXa activity of about 0.1 IU per 1 mL of the medium was confirmed. From this result, it was suggested that an antithrombin binding sequence was contained in the heparin-like sugar chain in the culture supernatant by the introduction of NDST2 and Hs3st1.

Although the present invention has been described in detail using specific embodiments, it will be apparent to those skilled in the art that various modifications and variations are possible without departing from the spirit and scope of the present invention. This application is based on a Japanese patent application filed on Oct. 2, 2019 (Application No. 2019-182467), the entire contents thereof being incorporated herein by reference.

1. A method for producing a heparin-like substance, the method comprising the following steps (1) to (3):
(1) preparing a mammalian cell that produces a heparin-like substance;
(2) preparing a recombinant cell in which a gene that encodes an extracellular domain of syndecan (hereinafter abbreviated as SDC) is introduced into the mammalian cell that produces a heparin-like substance and is prepared in step (1); and
(3) culturing the recombinant cell prepared in step (2) in a medium, and collecting a heparin-like substance from a resulting culture supernatant.

2. The production method according to claim 1, wherein the mammalian cell that produces a heparin-like substance is a cell into which at least one of a gene that encodes bifunctional heparan sulfate ester N-deacetylase/N-sulfate transferase (hereinafter abbreviated as NDST2) and a gene that encodes heparan sulfate ester glucosamine 3-sulfate transferase 1 (hereinafter abbreviated as Hs3st1) is introduced.

3. The production method according to claim 1, wherein the mammalian cell is a Chinese hamster-derived ovary (hereinafter abbreviated as CHO) cell.

4. The production method according to claim 2, wherein NDST2 is a protein comprising any one of the following amino acid sequences (1a) to (1c) and having a function of NDST2:
(1a) the amino acid sequence of SEQ ID NO: 2,
(1b) an amino acid sequence in which one to several amino acids are deleted, substituted or added in the amino acid sequence of SEQ ID NO: 2, and
(1c) an amino acid sequence having 80% or more homology with the amino acid sequence of SEQ ID NO: 2.

5. The production method according to claim 2, wherein Hs3st1 is a protein comprising any one of the following amino acid sequences (2a) to (2c) and having a function of Hs3st1:
(2a) the amino acid sequence of SEQ ID NO: 3,
(2b) an amino acid sequence in which one to several amino acids are deleted, substituted or added in the amino acid sequence of SEQ ID NO: 3, and
(2c) an amino acid sequence having 80% or more homology with the amino acid sequence of SEQ ID NO: 3.

6. The production method according to claim 1, wherein the extracellular domain of SDC is a protein comprising any one of the following amino acid sequences (3a) to (3c) and having a function of the extracellular domain of SDC:
(3a) the amino acid sequence of SEQ ID NO: 1,
(3b) an amino acid sequence in which one to several amino acids are deleted, substituted or added in the amino acid sequence of SEQ ID NO: 1, and
(3c) an amino acid sequence having 80% or more homology with the amino acid sequence of SEQ ID NO: 1.

7. The production method according to claim 2, wherein NDST2 and Hs3st1 are derived from a mouse or a human.

8. The production method according to claim 2, wherein the gene that encodes NDST2 comprises a base sequence that is any one of the following base sequences (1A) to (1D) and encodes an amino acid sequence of a protein having a function of NDST2:
(1A) the base sequence of SEQ ID NO: 4,
(1B) a base sequence in which one to several bases are deleted, substituted or added in the base sequence of SEQ ID NO: 4,
(1C) a base sequence which hybridizes under stringent conditions with a base sequence complementary to the base sequence of SEQ ID NO: 4, and
(1D) a base sequence having 80% or more homology with the base sequence of SEQ ID NO: 4.

9. The production method according to claim 2, wherein the gene that encodes Hs3st1 comprises a base sequence that is any one of the following base sequences (2A) to (2D) and encodes an amino acid sequence of a protein having a function of Hs3st1:
(2A) a base sequence of SEQ ID NO: 5,
(2B) a base sequence in which one to several bases are deleted, substituted or added in the base sequence of SEQ ID NO: 5,
(2C) a base sequence which hybridizes under stringent conditions with a base sequence complementary to the base sequence of SEQ ID NO: 5, and
(2D) a base sequence having 80% or more homology with the base sequence of SEQ ID NO: 5.

10. The production method according to claim 1, wherein the gene that encodes SDC comprises a base sequence that is any one of the following base sequences (3A) to (3D) and encodes an amino acid sequence of a protein having a function of the extracellular domain of SDC:
(3A) a base sequence of SEQ ID NO: 6,
(3B) a base sequence in which one to several amino acids are deleted, substituted or added in the base sequence of SEQ ID NO: 6,
(3C) a base sequence which hybridizes under stringent conditions with a base sequence complementary to the base sequence of SEQ ID NO: 6, and
(3D) a base sequence having 80% or more homology with the base sequence of SEQ ID NO: 6.

11. A recombinant cell in which a gene that encodes an extracellular domain of SDC is introduced into a mammalian cell that produces a heparin-like substance.

12. The recombinant cell according to claim 11, wherein the mammalian cell that produces a heparin-like substance is a cell into which at least one of a gene that encodes NDST2 and a gene that encodes Hs3st1 is introduced.

13. The recombinant cell according to claim 11, wherein the mammalian cell is a CHO cell.

14. A method for producing a cell that secretes and produces a heparin-like substance, the method comprising: introducing a recombinant expression vector comprising a gene that encodes an extracellular domain of SDC into a mammalian cell that produces a heparin-like substance.


SEQUENCE LISTING (7 sequences)

SEQ ID NO: 1 - protein, 229 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDR

SEQ ID NO: 2 - protein, 883 aa, Homo sapiens
MLQLWKVVRPARQLELHRLILLLIAFSLGSMGFLAYYVSTSPKAKEPLPLPLGDCSSGGAAGPGPARPPVPPRPPRPPETARTEPVVLVFVESAYSQLGQEIVAILESSRFRYSTELAPGRGDMPTLTDNTHGRYVLVIYENLLKYVNLDAWSRELLDRYCVEYGVGIIGFFRAHEHSLLSAQLKGFPLFLHSNLGLRDYQVNPSAPLLHLTRPSRLEPGPLPGDDWTIFQSNHSTYEPVLLASLRPAEPAVPGPVLRRARLPTVVQDLGLHDGIQRVLFGHGLSFWLHKLIFVDAVAYLTGKRLCLDLDRYILVDIDDIFVGKEGTRMKVADVEALLTTQNKLRTLVPNFTFNLGFSGKFYHTGTEEEDAGDDMLLKHRKEFWWFPHMWSHMQPHLFHNRSVLADQMRLNKQFALEHGIPTDLGYAVAPHHSGVYPIHTQLYEAWKSVWGIQVTSTEEYPHLRPARYRRGFIHNGIMVLPRQTCGLFTHTIFYNEYPGGSRELDRSIRGGELFLTVLLNPISIFMTHLSNYGNDRLGLYTFESLVRFLQCWTRLRLQTLPPVPLAQKYFELFPQERSPLWQNPCDDKRHKDIWSKEKTCDRLPKFLIVGPQKTGTTAIHFFLSLHPAVTSSFPSPSTFEEIQFFNSPNYHKGIDWYMDFFPVPSNASTDFLFEKSATYFDSEVVPRRGAALLPRAKIITVLTNPADRAYSWYQHQRAHGDPVALNYTFYQVISASSQTPLALRSLQNRCLVPGYYSTHLQRWLTYYPSGQLLIVDGQELRTNPAASMESIQKFLGITPFLNYTRTLRFDDDKGFWCQGLEGGKTRCLGRSKGRRYPDMDTESRLFLTDFFRNHNLELSKLLSRLGQPVPSWLREELQHSSLG

SEQ ID NO: 3 - protein, 311 aa, Homo sapiens [sequence omitted]

SEQ ID NO: 4 - DNA, 2652 bp, Homo sapiens
atgctccagttgtggaaggtggtacgcccagctcggcagctggaactgcaccgcctcatactgctgctgatcgctttcagcctgggctccatgggcttcctggcttattatgtgtccaccagccctaaggccaaggaacccttgcccctgcccttgggagactgcagcagcggtggggcagctggtcctggccctgcacggcctccagttccacctcggccccccaggcctccagagacagctcgaactgaacccgtggtccttgtgtttgtggagagtgcatactcacagctggggcaggaaattgtggccatcctggagtctagtcgttttcgttatagcactgagttggcacctggccgaggggacatgcccacattgactgataatacccatggccgctatgtcttggtcatttatgagaacctgctcaagtatgtcaacctggatgcctggagtcgggaactgctagaccggtactgcgtggagtatggtgtgggcatcattggctttttccgagcccacgagcacagcctactgagcgcccagctcaagggctttcccctttttttacactcaaacttggggctccgggactaccaagtgaatccttctgccccgctactgcatctcacacgccccagccgcctagaaccagggccactgcctggtgatgactggaccatcttccaatccaatcatagtacatatgaaccagtgcttcttgccagccttcggccagctgagcccgcagtgccaggaccagttcttcgtcgggcccggcttcccactgtggtacaggacctggggcttcatgatggcatccagcgggtgctctttggacatggcctttccttctggctccacaaacttatcttcgttgatgctgttgcatacctcactggcaagcgcctctgcctggaccttgaccgctacatcttggtagacatcgatgacatctttgtgggcaaggaagggacccgcatgaaggtggctgatgttgaggctctgttgaccacccagaacaaactcaggaccttagttcccaacttcaccttcaacttgggcttctcgggcaagttctatcatactgggacagaggaggaggatgcaggggacgacatgctgctgaagcaccgcaaagagttctggtggttcccccacatgtggagccacatgcagccacacctgttccacaatcgctccgtgctggctgaccagatgaggctcaacaaacagtttgctctggagcatgggattcccacggacctggggtatgctgtggccccccaccactcgggtgtgtaccccatccacacgcagctctatgaggcctggaaatccgtgtggggcatccaggtgaccagcactgaggagtatccccatctccgccctgcccgctaccgccgtggcttcattcacaatggcattatggtgctgccccggcagacatgtggcctcttcactcacacaatcttctataatgagtatcctggaggctctcgtgaactagaccggagcatccgaggtggagagctctttctgacagtgctgcttaatccgatcagcatctttatgacccatctgtccaattatggaaatgaccggctgggcctatacacctttgagagcttggtgcgcttcctccagtgttggacacggctgcgcctacagacccttcctcctgtcccacttgcacagaagtactttgaacttttccctcaggagcgaagccccctttggcagaatccctgtgatgacaagaggcacaaagatatctggtccaaggagaaaacctgtgatcgtctcccgaagttcctcattgtgggaccccagaaaacagggactacagctattcacttcttcctgagcctgcacccagctgtaactagcagcttccctagccccagcacatttgaggagattcagttcttcaacagccctaattaccacaagggtattgactggtacatggatttcttccctgttccttccaatgccagcactgatttcctatttgaaaaaagtgccacctactttgactctgaagttgtaccacggcggggggctgccctcctgccacgagccaagatcatcacagtgctcaccaaccctgctgacagggcctactcctggtaccagcatcagcgagcccatggagacccagttgctctgaactataccttctatcaggtgatttcagcctcctcccagacccctctggcactacgctccctgcagaaccgctgtcttgtccctggctactattctacccatctacaacgctggctgacttactacccctctggacagttgctgattgtggatgggcaagagctgcgtaccaacccagcagcctcaatggagagcatccagaagttcctgggtatcacaccctttctgaactacacacggaccctcaggtttgatgatgataagggattttggtgccagggacttgaaggtggtaagactcgctgtctaggccggagcaaaggccggaggtatccagatatggacactgagtcccgtcttttccttacggattttttccggaaccataatttggagttgtcgaagctgctgagccggcttggacagccagtgccctcgtggcttcgggaagaactgcagcattccagtctgggctga

SEQ ID NO: 5 - DNA, 936 bp, Mus musculus
atgaccttgctgctcctgggtgcggtgctgctggtggcccagccccagcttgtgcattcccacccggctgctcctggcccggggctcaaacagcaggagcttctgaggaaggtgattattctcccagaggacaccggagaaggcacagcatccaatggttccacacagcagctgccacagaccatcatcattggggtgcgcaagggtggtacccgagccctgctagagatgctcagcctgcatcctgatgttgctgcagctgaaaacgaggtccatttctttgactgggaggagcattacagccaaggcctgggctggtacctcacccagatgcccttctcctcccctcaccagctcaccgtggagaagacacccgcctatttcacttcgcccaaagtgcctgagagaatccacagcatgaaccccaccatccgcctgctgcttatcctgagggacccatcagagcgcgtgctgtccgactacacccaggtgttgtacaaccaccttcagaagcacaagccctatccacccattgaggacctcctaatgcgggacggtcggctgaacctggactacaaggctctcaaccgcagcctgtaccatgcacacatgctgaactggctgcgttttttcccgttgggccacatccacattgtggatggcgaccgcctcatcagagaccctttccctgagatccagaaggtcgaaagattcctgaagctttctccacagatcaacgcctcgaacttctactttaacaaaaccaagggcttctactgcctgcgggacagtggcaaggaccgctgcttacacgagtccaaaggccgggcgcacccccaggtggatcccaaactacttgataaactgcacgaatactttcatgagccaaataagaaatttttcaagctcgtgggcagaacattcgactggcactga

SEQ ID NO: 6 - DNA, 687 bp, Homo sapiens
atgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgc

SEQ ID NO: 7 - DNA, 477 bp, Homo sapiens
atgatgcagaagctactcaaatgcagtcggcttgtcctggctcttgccctcatcctggttctggaatcctcagttcaaggttatcctacgcggagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagacagccaggacttgggtcaacatggattagaagaggattttatgttataa